(19) United States
(12) Patent Application Publication
(10) Pub. No.: US 2024/0238484 A1
(43) Pub. Date: Jul. 18, 2024

(54) Composite gel for removing residual stone fragments after lithotripsy

(71) Applicant: Xiaochen Zhou, Nanchang, Jiangxi (CN)

(72) Inventors: Xiaochen Zhou, Nanchang, Jiangxi (CN); Renrui Kuang, Nanchang, Jiangxi (CN); Haibo Xi, Nanchang, Jiangxi (CN); Yue Yu, Nanchang, Jiangxi (CN); Jun Deng, Nanchang, Jiangxi (CN); Yujun Chen, Nanchang, Jiangxi (CN); Xinpeng Chen, Nanchang, Jiangxi (CN)

(21) Appl. No.: 18/562,323

(22) PCT Filed: May 12, 2022
(86) PCT No.: PCT/CN2022/092370

§ 371 Date: Nov. 19, 2023

(30) Foreign Application Priority Data
May 19, 2021 (CN) 202110547279.5
May 1, 2022 (CN) 202210491708.6

(51) Int. Cl. A61L 31/04

(52) U.S. Cl. CPC A61L 31/047; A61L 31/046; A61L 2300/442; A61L 2300/45

(57) ABSTRACT

A composition gel for surgical efficiency improvement of urinary stone lithotripsy and stone fragments removal in renal collecting system includes component A and component B. The component A contains fibrinogen and fibrin stabilizing factors. The component B contains thrombin. Moreover, at least one of the components A and B contains Ca²⁺, plasminogen and a coloring agent. The composition gel may quickly form plasminogen-containing fibrin gel with color, and certain toughness and plasticity in urine and normal saline environments, may adhere to and wrap stone fragments in such environments, and is removed by negative pressure suction and/or stone basket. The composition gel is suitable for efficiently removing stone fragments which remain after lithotripsy and are unable to be removed by an effective method at present. It has good biosafety, and achieves stone clearance rate of more than 85% without damage to the kidney and surgical instruments. The fibrin gel may be naturally dissolved in urine without the risk of urinary system obstruction.

### BACKGROUND OF THE PRESENT INVENTION

### Field of Invention

The present invention relates to a composite gel for improving the surgical efficiency of urinary stone lithotripsy, and assisting the clearance of stone fragments in the renal collecting system.

### Description of Related Arts

Urinary calculus is a benign disease of the urinary system. However, it may lead to urinary tract obstruction, infection, pain, and accompanied by the risk of inducing malignant transformation of urinary tract epithelium. Therefore, it is an important disease that threatens human health.

Up to date, ESWL (extracorporeal shockwave lithotripsy), ureteroscopic lithotripsy and PCNL (percutaneous nephrolithotomy) are the three major treatments for urinary calculi. Flexible ureteroscope was developed in 1964. Professor Marshall was the first in history who attempted to retrogradely enter the renal collecting system through the urethra and bladder and perform lithotripsy using using flexible ureteroscope. The surgery was known as retrograde intrarenal surgery (RIRS). Since 1995, holmium laser combined with flexible ureteroscope were used in the treatment of renal and ureteral stones, urinary tract obstruction and tumors, which marked the beginning of the application of flexible ureteroscope in clinical settings. Flexible ureteroscope has been widely used in the diagnosis and treatment of upper urinary tract diseases since then. Compared with traditional rigid ureteroscope, flexible ureteroscope is composed by the passively bendable body segment and a head segment that is able to bend by 270-degree passively and actively. As such, flexible ureteroscope is able to examine a much wider range of space and can almost reach the whole collecting system. Compared with PCNL, passing through natural orifices and body cavity, flexible ureteroscopic surgery yielded no extra trauma to the renal parenchyma. Therefore, flexible ureteroscopic surgery is accompanied by much lower risk of bleeding and a wider range of inspection. Flexible ureteroscope is now considered as the first-line choice for surgical treatment of renal and ureteral stones that are less than 2 cm.

With the development of lithotripsy energy platforms compatible with RIRS, improvement of surgical techniques and better understanding of the key elements of RIRS, the surgical indications of RIRS are continuously expanding. Some scholars have reported that stones>2.0 cm or even staghorn stones, complicated stones (solitary kidney, horseshoe kidney and spinal deformity) and pediatric urinary stones can be safely and effectively managed by RIRS. RIRS has been widely used in China in the recent 10 years, and become an important surgical technique for the treatment of urinary stones.

At present, the standard protocol for RIRS lithotripsy includes a series of steps: grabbing and moving the target stone to a single renal calyx (generally a middle or upper calyx with small calyx neck and large space inside the calyx for fully accommodating the target stone) by a stone basket, performing laser lithotripsy (fragmentation or pop-dusting), and removing large stone fragments by stone basket. During the process of basket extraction of stone fragments, supplemental lithotripsy is performed if there are any fragments that are too large to be extracted. For fragments that are too small to be extracted by basket, pop-dusting is performed (pop-dusting: continuous activation of laser power while the tip of the laser fiber is placed in a relatively safe central zone where most fragments resides. Fragments in the calyx will randomly jump to the tip of the laser by water flow and laser-generated kinetic energy, and be subjected to laser induced fragmentating and dusting). small stone fragments (which are usually <2-3 mm) and dust generated by pop-dusting are expected to be spontaneously passed out with the assistance of increasing water intake, medications, special position and other measurements. Limited by surgical instruments and techniques, the risk of residual stones after RIRS cannot be ignored. It has been reported that the chances of having residual stone<3 mm, <2 mm and <1 mm were 10-15%, 16.1% and as high as 86%, respectively.

With the continuous improvement of people's health awareness, there are fewer and fewer patients with stone burdens that are too large to be managed by RIRS. Meanwhile, with the popularization of RIRS instruments and the improvement of RIRS techniques, the proportion of RIRS in the treatment of urinary stones is increasing year by year. However, as mentioned above, due to the limitations of technology and instruments, it is often difficult to avoid residual stones after RIRS, and even there are often more residual small stone fragments and powders, which require to be excreted by patients themselves after surgery. Unfortunately, although most of the residual stones in patients after RIRS are able to be excreted by patients themselves, the residual stones are excreted by patients themselves for a long time, and about 20-30% of patients are still unable to excrete the residual stones and have an aggravation of stones. In a recent study, 148 patients with postoperative residual stones are followed up for 2 years, in which the average self-excretion time for <1 mm of residual stones is 9 months, 18.1% of the 148 patients have an aggravation of stones; the average self-excretion time for 1-3 mm of residual stones is 13.9 months, and 28.6% of the 148 patients have an aggravation of stones. Another study of 142 patients with residual stones after flexible lithotripsy followed up for about 54 months shows that the self-excretion rate of 1-3 mm of residual stones is only 30.23%. These investigations show that the residual stones are excreted by patients themselves for a long time, and there are still more patients who are unable to excrete the residual stones and have an aggravation of stones. Therefore, it is of great significance to avoid residual stones after RIRS, but there is no effective method or preparation that is able to efficiently remove small stone fragments after RIRS.

### SUMMARY OF THE PRESENT INVENTION

### (I) Technical Problems to be Solved

Aiming at problems in the prior art, the present invention provides a composition gel for removing residual stone fragments after lithotripsy, which helps to significantly improve the outcome of patients with stones after RIRS, and has important scientific significance and popularization value.

### (II) Technical Schemes

To achieve the above object, the present invention adopts technical schemes as follows.

A composition gel for removing residual stone fragments after lithotripsy comprises component A and component B, wherein:
the component A contains fibrinogen and a fibrin stabilizing factor;
the component B contains thrombin;
at least one of the component A and the component B contains Ca²⁺.

Preferably, the at least one of the component A and the component B contains plasminogen.

According to a preferred embodiment of the present invention, the at least one of the component A and the component B contains a non-cyto toxic colouring agent for convenience of visualization and observation.

The colouring agent, Ca²⁺ and plasminogen are able to be added separately to the component A, or be added separately to the component B, or be added together to the at least one of the components A and B. The component A acts as a main body in the composition gel, the component B acts as a catalyst in the composition gel, and the composition gel with wrapping and adhesion ability is formed after mixing the components A and B.

According to another preferred embodiment of the present invention, the colouring agent is methylene blue, chlorophyll or indocyanine green.

According to another preferred embodiment of the present invention, both the fibrinogen and the fibrin stabilizing factor are derived from human or animal blood or blood products. Specifically, both the fibrinogen and the fibrin stabilizing factor of the component A are derived from human blood, pig blood, cow blood or sheep blood.

According to another preferred embodiment of the present invention, the component A further contains normal saline, wherein the fibrinogen and the fibrin stabilizing factor are diluted in the normal saline.

According to another preferred embodiment of the present invention, the component A contains fibrinogen, a fibrin stabilizing factor, methylene blue and normal saline, wherein a concentration of the methylene blue is in a range of 20.0 to 60.0 μg/ml.

According to another preferred embodiment of the present invention, both the thrombin and the plasminogen are derived from human or animal blood or blood products.

Specifically, the thrombin of the component B is one of human thrombin, pig thrombin, cow thrombin and sheep thrombin; as long as the thrombin of the component B corresponds to the source of the fibrinogen and the fibrin stabilizing factor of the component A, the thrombin of the component B meets operation requirements. In the case of source correspondence, the thrombin is able to promote the conversion of the fibrinogen into the composition gel.

According to another embodiment of the present invention, the component B further contains normal saline, wherein the thrombin is diluted in the normal saline.

According to another embodiment of the present invention, the component B contains thrombin, Ca²⁺, plasminogen and normal saline, wherein a concentration of the plasminogen is in a range of 0.1 to 1 mg/ml.

Preferably, the plasminogen is derived from one of human blood, pig blood, cow blood and sheep blood, and corresponds to the source of the fibrinogen and the fibrin stabilizing factor of the component A.

The present invention has technical effects as follows.

The composition gel provided by the present invention is used to efficiently remove small stone fragments in the kidney, ureter or bladder. Before preparing the composition gel, the component A and the component B are separately prepared or packaged. During preparing the composition gel, the components A and B are simultaneously injected into an area of stones in the kidney, ureter or bladder through a device with ejection function (which are able to be injected immediately after evenly mixing or mixed naturally after injection), so that the composition gel wraps and adheres to the stones. The composition gel adhering to and wrapping the stones is removed through the UAS (ureteral access sheath) by negative pressure suction and/or a stone extractor.

The composition gel provided by the present invention has characteristics as follows.

(1) The components A and B of the composition gel are able to quickly form the fibrin gel containing plasminogen with a certain toughness and plasticity in the environment of normal saline. The formed fibrin gel is able to adhere to and wrap small stone fragments in the environment of normal saline. The fibrin gel adhering to and wrapping the small stone fragments has sufficient softness, plasticity and toughness, and is removed from the human body by negative pressure suction and/or a stone extractor. The composition gel provided by the present invention is especially suitable for efficiently removing small stone fragments which remain after lithotripsy, has good biosafety, and achieves a stone clearance rate of more than 85% without any damage to the collection system and surgical instruments. The composition gel provided by the present invention is configured to remove small stone fragments after lithotripsy of the urinary system.

(2) The composition gel provided by the present invention contains the colouring agent that is non-toxic to human cells. The colouring agent is helpful to inject the composition gel into the designated area under direct vision, providing good visibility and facilitating stone removal operations. The shade of colour of the fibrin gel is related to the concentration of the colouring agent such as methylene blue. The user is able to adjust the shade of colour of the fibrin gel by adjusting the concentration of the colouring agent according to the actual needs.

(3) The softness, plasticity and toughness of the fibrin gel formed by the composition gel are related to the concentrations of fibrinogen and the fibrin stabilizing factor of the component A (which acts as the main body in the composition gel), and the concentrations of thrombin and Ca²⁺ of the component B (which acts as the catalyst in the composition gel). Therefore, in actual use, they are able to be adjusted according to the demand or the instrument for removing residual stone fragments.

(4) In the present invention, at least one of the components A and B contains plasminogen, so that the fibrin gel formed by the components A and B is naturally dissolved in urokinase-saline solution or healthy human urine, reducing or eliminating the risk of urinary obstruction caused by the fibrin gel.

The fibrin gel provided by the present invention is able to assist in removing small stone fragments in the renal collecting system, ureter and bladder with a variety of urinary endoscopes, which significantly improves the stone clearance rate of stone surgery of the urinary system, reduces the risk of postoperative stone recurrence, and has important clinical popularization value.

### BRIEF DESCRIPTION OF THE DRAWINGS

FIGS. 1A and 1B are photos showing that a composition gel provided by the present invention forms gel in normal saline.

FIGS. 1C-1E are experimental photos showing that when two components of the composition gel are diluted 25-35 times, the formed translucent gel is able to adhere to and wrap stone fragments, and the gel adhering to and wrapping the stone fragments is easily removed from a human body through a 12 Fr of UAS (ureteral access sheath) by negative pressure suction.

FIG. 2A is a diagram of connection of a flexible ureteroscope and other elements.

FIG. 2B shows that after simultaneously rapidly injecting the component A and the component B (blue) into renal calyces where the stone fragments are located through an operating passage of the flexible ureteroscope, 1 ml of normal saline is injected.

FIG. 2C shows the gel wrapping the stone fragments after waiting for 3-5 seconds.

FIG. 3A shows a metallic mesh screening with a mesh size of 1 mm.

FIG. 3B shows a metallic mesh screening with a mesh size of 2 mm.

FIG. 3C shows a metallic mesh screening with a mesh size of 3 mm.

FIG. 3D shows ≤1 mm of stone fragments are selected from naturally dried and ground stone specimens by the metallic mesh screening with the mesh size of 1 mm.

FIG. 3E shows ≤2 mm of stone fragments are selected from naturally dried and ground stone specimens by the metallic mesh screening with the mesh size of 2 mm.

FIG. 3F shows ≤3 mm of stone fragments are selected from naturally dried and ground stone specimens by the metallic mesh screening with the mesh size of 3 mm.

FIG. 4 shows that the gel, formed by the undiluted two components and the dilute solutions of two components with different dilution multiples in normal saline, is naturally dissolved in healthy human urine and normal saline.

FIG. 5 shows a process of constructing a model of an ex-vivo pig kidney with human stones.

FIG. 6 shows the removed gel wrapping the stone fragments after performing flexible ureteral lithotomy on the model of the ex-vivo pig kidney with the human stones, wherein FIG. 6A shows that the gel is removed from the human body via the UAS through the operating passage of the flexible ureteroscope by negative pressure suction; FIG. 6B shows that the gel wrapping the stone fragments is directly removed from the human body through the UAS by negative pressure suction; and FIG. 6C shows that the gel wrapping the stone fragments is removed by a stone extractor.

FIG. 7 shows the gel formed by adding different final concentrations of methylene blue into the component B in a white background state and in a transparent state.

FIG. 8A is a plain abdominal radiograph of a patient with right ureteral stones.

FIG. 8B shows a CT (computed tomography) image of a patient's hypogastrium that the composition gel provided by the present invention is applied after flexible ureteral lithotripsy, in which no obvious residual stone fragments are found.

FIG. 9 shows a process of performing flexible ureteral lithotripsy on the patient, wherein FIG. 9A shows that the 12/14 Fr of UAS is indwelled by conventional method, intrarenal conditions are explored by the flexible ureteroscope, the stones are located and pulverized by laser; FIG. 9B is a photograph showing that the stones are pulverized into <2 mm of stone fragments; FIG. 9C shows that the dilute solutions of the components A and B are simultaneously rapidly injected into the designated area; FIG. 9D is an image showing that the position of the gel is observed; FIG. 9E shows a process of removing the gel with an NCircle® stone extractor; and FIG. 9F shows that the composition gel adhering to a large amount of stone fragments is removed from the human body during operation.

### DETAILED DESCRIPTION OF THE PREFERRED EMBODIMENT

In order to better understand the present invention, the present invention is described in detail by specific embodiments in combination with accompanying drawings.

A basic technical scheme of the present invention is as follows. A composition gel for removing residual stone fragments after lithotripsy comprises component A and component B both of which are separately prepared or packaged, wherein the component A contains fibrinogen and a fibrin stabilizing factor; the component B contains thrombin; at least one of the component A and the component B contains Ca²⁺ and plasminogen; each ingredient in the component A and each ingredient in the component B need to be diluted with normal saline to a certain extent for use.

In order to achieve visualization, at least one of the component A and the component B contains a non-cyto toxic colouring agent such as methylene blue. Of course, the methylene blue is able to be replaced with chlorophyll or indocyanine green. A concentration of the colouring agent determines a color of the gel, so that the concentration of the colouring agent is adjusted for meeting the visualization requirement. If the color of the gel is too dark, it is difficult to observe the situation that the stone fragments are wrapped by the gel, and if the color of the gel is too light, it is difficult for the gel to be visually observed. Preferably, when the component B contains the methylene blue, the concentration of the methylene blue in normal saline is in a range of 20.0 to 60.0 μg/ml.

During preparing the gel, the components A and B are simultaneously injected into an area of stones in the kidney, ureter or bladder through a device with ejection function. A fibrin gel with a certain softness, plasticity and toughness is quickly formed after mixing the components A and B. After the fibrin gel adheres to and wraps stone fragments, the fibrin gel adhering to and wrapping the stone fragments is removed from the human body by negative pressure suction and/or a stone extractor, so as to achieve the purpose of removing the stone fragments.

After the fibrinogen and the fibrin stabilizing factor of the component A are mixed with the thrombin and Ca²⁺ of the component B, a fibrin gel with a certain toughness and plasticity, and capable of adhering to and wrapping stones is quickly formed in the environment of normal saline and urine. As a widely used chromogenic agent in clinical practice, the methylene blue contained in the component A is able to effectively highlight the position of the fibrin gel in normal saline and urine for observation and operation, without affecting the display of the stones adhered and wrapped by the fibrin gel. The plasminogen of the component B is able to be activated by the thrombin in the catalyst to form fibrinolytic enzyme capable of dissolving the fibrin gel, without affecting the quick formation of the fibrin gel. Also, the plasminogen of the component B is able to be activated by urokinase in the urine to form fibrinolytic enzyme with the activity of hydrolyzing the fibrin gel, which helps to accelerate the dissolution and excretion of unexpected residual fibrin gel in the human body.

The softness, plasticity and toughness of the gel are directly related to the concentration of the fibrinogen and the fibrin stabilizing factor of the component A, and the concentration of the thrombin and Ca²⁺ of the component B. Therefore, during practical applications, the softness, plasticity and toughness of the gel are able to be adjusted according to actual needs or the specific instrument for removing residual stone fragments.

Preferably, both the fibrinogen and the fibrin stabilizing factor are derived from human or animal blood or blood products. Specifically, both the fibrinogen and the fibrin stabilizing factor are derived from human blood, pig blood, cow blood or sheep blood.

Preferably, both the thrombin and plasminogen are derived from human or animal blood or blood products. Specifically, the thrombin of the component B is one of human thrombin, pig thrombin, cow thrombin and sheep thrombin; as long as the thrombin of the component B corresponds to the source of the fibrinogen and the fibrin stabilizing factor of the component A, the thrombin of the component B meets operation requirements. In the case of source correspondence, the thrombin is able to promote the conversion of the fibrinogen into the gel.

Specifically, the plasminogen is derived from one of human blood, pig blood, cow blood and sheep blood; as long as the plasminogen of the component B corresponds to the source of the fibrinogen and the fibrin stabilizing factor of the component A, the plasminogen of the component B meets operation requirements.

The function and principle of each ingredient in the composition gel provided by the present invention are described as follows.

The fibrinogen of the component A which acts as the main body of the gel: fibrinoge is the precursor of fibrin, is mainly synthesized by human and animal liver cells and is the most abundant blood coagulation factor in plasma; fibrinogen, with a molecular weight of about 340 kDa, is a triplet spherical protein composed of α, β and γ polypeptide chains; fibrinogen forms fibrin monomer under the action of blood coagulation factors such as thrombin, fibrin stabilizing factor and Ca²⁺, and covalently combines with each other to form fibrin polymers; its α-chains interlace, overlap and covalently cross-link to form a stable fibrin network for forming the gel; when the concentration of other ingredients is determined, the toughness and plasticity of the gel depend on the concentration of fibrinogen.

If the gel has low softness and poor plasticity, it is difficult to deform and is unable to be removed from the human body by negative pressure suction; if the gel has low toughness, it is easy to break in the process of negative pressure suction, so that it is unable to effectively “wrap and grab stone fragments”.

The fibrin stabilizing factor of the component A which acts as the main body of the gel: the fibrin stabilizing factor, also known as blood coagulation factor XIII (FXIII), is a glycoprotein synthesized in the bone marrow and liver of humans and animals; the fibrin stabilizing factor, with a molecular weight of about 340 kDa, is a tetramer glycoprotein composed of two catalytic A subunits (FXIIII-A) and two carrier B subunits (FXIIIB); the fibrin stabilizing factor is involved in the formation of thrombin, and is able to cross-link the α-chain and γ-chain of fibrin, which contributes to the rapid formation of fibrin network, namely, producing gel) and resistance to fibrinolysis, so that the gel has strong toughness.

Methylene blue: methylene blue is an aromatic heterocyclic compound; its chemical name is 3, 7-bis (dimethylamino) phenothiazine-5-onide chloride; its chemical formula is C₁₆H₁₈N₃ClS, its CAS accession number is 61-73-4; methylene blue is soluble in water; methylene blue is widely used as a chemical indicator, dye, biological colouring agent and clinical drug, and is also used clinically to treat urinary stones, occlusive arterial disease and neurodermatitis; the aqueous solution of methylene blue is blue in normal saline, and is able to be reduced to colorless state in the presence of reducing agents such as ammonia.

The thrombin of the component B which acts as the catalyst of the gel: thrombin, with a molecular weight of about 37 kDa, is a proteolytic enzyme formed after the activation of prothrombin (blood coagulation factor II); it is composed of two peptide chains with molecular weights of 31 kDa and 6 kDa, respectively through disulfide bonds; thrombin catalyzes fibrinogen to form fibrin monomers, and is also able to activate the fibrin stabilizing factor (XIII) to change into XIIIa, so that the fibrin monomers are connected with each other to form water-insoluble fibrin polymers, and are interwoven with each other into a network, thus the gel with a certain flexibility and plasticity is obtained; and moreover, thrombin also has the effect of activating plasminogen.

The calcium ion (Ca²⁺) of the component B which acts as the catalyst of the gel: Ca²⁺ is an indispensable cation in various blood coagulation pathways in vivo; in the blood coagulation intrinsic pathway, Ca²⁺ is able to help to activate the factor XI, all of the activated factor XI, the factor VIII and the activated factor IX activate the factor X together; in the blood coagulation extrinsic pathway, Ca²⁺, and the factors III and VII activate the factor X together; in the common blood coagulation pathway, Ca²⁺ is able to convert fibrinogen into fibrin monomers together with the factor V and the activated factor X; and in addition, Ca²⁺ is also able to assist in the activation of the fibrin stabilizing factor, and continue to assist the fibrin stabilizing factor to convert soluble fibrin monomers into stable fibrin polymers, so Ca²⁺ is mainly used to promote the rapid formation of the gel.

The plasminogen of the component B which acts as the catalyst of the gel: plasminogen or profibrinolysin, also known as plasmatrypsinogen, is an inactive precursor of plasmin; plasminogen directly becomes plasmin through the activation of tissue activators and urinary activators (urokinase); plasmin is a proteolytic enzyme that is able to dissolve fibrin clots (gels); the addition of plasminogen helps to naturally dissolve the residual gel after removing stone fragments in the urine, reducing or eliminating the risk of urinary tract obstruction.

Also, the component B which acts as the catalyst of the gel does not contain the above plasminogen.

In order to further verify the technical effect of the present invention, the present invention is explained detailedly in combination with the following embodiments.

### First Embodiment

Referring to FIGS. 1A and 1B, normal saline and a certain amount of naturally air-dried and ground human stones are put into a vial; and then, a certain amount of component A containing fibrinogen and fibrin stabilizing factor and a certain amount of component B (containing thrombin, Ca²⁺ and plasminogen) are evenly mixed and injected into the vial with normal saline through a special injection hose, so that a tough, milky fibrin gel is formed within seconds.

In the vial, a concentration of the fibrinogen is 2.2 mg/ml, a concentration of the fibrin stabilizing factor is 1.6 mg/ml, a concentration of the thrombin is 20 IU/ml, a concentration of Ca²⁺ is 5 mM, and a concentration of the plasminogen is 1 mg/ml.

### Second Embodiment

This embodiment mainly studies the effect of dilution multiple on the plasticity and toughness of the gel. The experimental process comprises steps of:
(1) preparing an undiluted stock solution of the component A and an undiluted stock solution of the component B, wherein:
the component A acts as a main body of the gel, and the component B acts as a catalyst;
in the component A, a concentration of the fibrinogen is 2.2 mg/ml, a concentration of the fibrin stabilizing factor is 1.6 mg/ml; in the component B, a concentration of the thrombin is 20 IU/ml, a concentration of Ca²⁺ is 5 mM, and a concentration of the plasminogen is 1 mg/ml;
in the component A, a total concentration of the fibrinogen and fibrin stabilizing factor is in a range of 1.8 to 3.8 mg/ml; in the component B, a concentration of the thrombin is in a range of 15.0-21.7 IU/ml;
(2) diluting the undiluted stock solutions of the components A and B 1.414, 2, 2.828, 4, 5.656, 8, 11.312, 16, 22.624, 32, 45.248, 64, 90.496, 128, 181.047 and 256 times with a first amount of normal saline by a wide range of proportional dilution method, respectively;
(3) putting 10 mL of normal saline and a certain amount of naturally air-dried and ground human stones into a 30 mL of vial;
(4) obtaining a gel by simultaneously injecting the dilute solutions of the components A and B at a same dilution multiple into the vial, and performing negative pressure suction on the gel by a 12 Fr of UAS (ureter access sheath), wherein:
experimental results show that the fibrin gel, formed by the undiluted stock solutions of the components A and B, is tough, is able to adhere to and wrap the human stones, and is difficult to be removed from a human body by the 12 Fr of UAS; and however, the fibrin gel, formed by the dilute solutions of the components A and B with a dilution multiple of 32 times, has good toughness and plasticity, is able to adhere to and wrap the human stones, and is able to be removed from the human body by the 12 Fr of UAS; the fibrin gel, formed by the dilute solutions of the components A and B with a dilution multiple of 64 times or higher, is unable to adhere to and wrap the human stones effectively; and
(5) diluting the undiluted stock solutions of the components A and B 20, 25, 30, 35, 40 and 45 times with a second amount of normal saline by a small range of isometric dilution method, respectively; obtaining a gel by simultaneously injecting the dilute solutions of the components A and B at the same dilution multiple into the vial; and performing negative pressure suction on the gel by the 12 Fr of UAS, wherein:
experimental results are shown in FIGS. 1C to 1E that referring to FIG. 1C, the gel, which is translucent, is formed within 3-5 seconds by the dilution solutions of the components A and B with the dilution multiple in a range of 25 to 35 times; referring to FIG. 1D, the gel is able to adhere to and wrap stone fragments, has excellent plasticity and toughness, and is able to be easily removed from the human body by negative pressure suction through the 12 Fr of UAS, wherein a tip of the UAS does not need to contact the human stones, and is about 0.5 cm away from the human stones deposited at a bottom of the vial, and also, the gel is able to be grabbed out of the human body with a stone extractor; referring to FIG. 1E, after the human stones and the gel are removed, it is still visible that normal saline with a thickness of about 0.5 cm remains at the bottom of the vial.

### Third Embodiment

This embodiment mainly studies the effect of injection method on the performance of lithotomy with gel. The injection methods are as follows.

The first injection method comprises successively injecting the dilution solutions of the components A and B with a dilution multiple of 32 times according to the second embodiment of the present invention, into a specified liquid environment.

The second injection method comprises injecting after evenly mixing the dilution solutions of the components A and B with the dilution multiple of 32 times according to the second embodiment of the present invention, into a specified liquid environment within 3 seconds.

The third injection method comprises simultaneously injecting the dilution solutions of the components A and B (added with methylene blue) with the dilution multiple of 32 times according to the second embodiment of the present invention, into a designated area through an operating passage of a flexible ureteroscope with three three-way stopcocks; and then adding a small amount of normal saline.

Experimental results show that:

The first injection method, that the components are successively injected into the specified liquid environment, may result in the rapid dilution of one component which is injected first in the liquid environment, and thus the gel with expected physical properties is unable to be formed.

For the second injection method, although it takes about 3-5 seconds for the dilute solutions of the components A and B to form the gel after evenly mixing, it is easy to block the lumen in practical applications if the dilute solutions of the components A and B are injected into the designated area within 3 seconds after evenly mixing. A total length of the ureter in adults is about in a range of 25 to 35 cm, a length of the urethra in men is about in a range of 20 to 22 cm and a length of the urethra in women is about in a range of 4 to 6 cm. It is necessary to place the UAS at ureteropelvic junction instead of external orifice of urethra, so as to perform relatively safe lithotripsy on kidney stones, thus ensuring the smooth progress of RIRS (retrograde intrarenal surgery). Due to the anatomical differences between men and women and the variability of the distance from the external orifice of male urethra to the internal orifice of male urethra, the length of the used UAS is generally 46 cm (male) and 36 cm (female), and the inner circumference thereof is generally 12 Fr (which is equivalent to the diameter of about 3.82 mm). Therefore, the volume of the lumen of the UAS is about 5.27 ml (male) and 4.12 ml (female). The flexible ureteroscope goes into the kidney through the UAS. At present, the length of the flexible ureteroscope is generally 60 cm, the outer circumference of the flexible ureteroscope is generally in a range of 8 to 9 Fr (which is equivalent to the diameter of the flexible ureteroscope in a range of about 2.55 to 2.87 mm), the inner circumference of the operating passage of the flexible ureteroscope is generally in a range of 3.5 to 4 Fr (which is equivalent to the diameter of the operating passage of the flexible ureteroscope in a range of about 1.11 to 1.27 mm). Therefore, the volume of the operating passage of the flexible ureteroscope is in a range of about 0.58 to 0.76 ml. The volume of the renal pelvis in normal adults is in a range about 3 to 10 ml (average 7.5 ml), and the volume of the renal calyx is even smaller. If the dilute solutions of the components A and B are injected into the renal pelvis through the lumen of the UAS after evenly mixing, a relatively large amount of gel remain in the lumen (male: 5.27 ml, female: 4.12 ml), so that additional normal saline needs to be injected for flushing the residual gel out of the lumen to avoid blockage. However, the lumen of the UAS is too large, so there is also the possibility that the residual gel is unable to be completely flushed out of the lumen by normal saline. As a result, the method that the dilute solutions of the components A and B are injected through the lumen of the UAS for forming the gel is not advisable. If a microcatheter with an inner circumference of 2 Fr and an outer circumference of 3 Fr is placed into the operating passage of the flexible ureteroscope for achieving intrarenal injection of two components of the gel, the possibility of the gel blocking the operating passage of the flexible ureteroscope is avoided, and however, the surgery cost is increased, and the surgery time is also increased due to repeated entry and exit of the microcatheter.

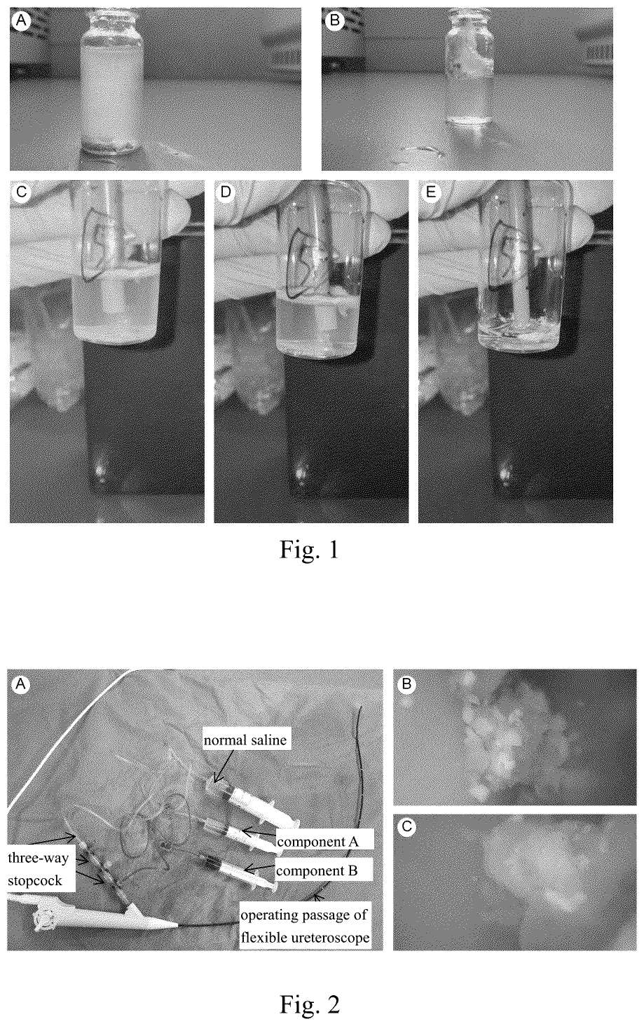

Referring to FIG. 2, three three-way stopcocks are connected with three syringes respectively, wherein there is normal saline in a top syringe, there is the component A in a middle syringe, and there is the blue component B in a bottom syringe; the three three-way stopcocks are connected with each other in series; an end of one of the three three-way stopcocks which is connected with the top syringe is connected with the operating passage of the flexible ureteroscope, such that the components A and B of the gel are simultaneously injected through the operating passage of the flexible ureteroscope; and then a small amount of normal saline, which exceeds the volume of the operating passage of the flexible ureteroscope, namely, 0.58 to 0.76 ml, is injected, such that the components A and B of the gel are injected into the designated area, the gel is formed in the liquid environment of normal saline, and the operating passage of the flexible ureteroscope is not blocked. Moreover, even if no additional normal saline is injected for flushing the lumen, it is also convenient to use a guidewire or a stone extractor to dredge the lumen.

FIG. 2A is a diagram of connection of a flexible ureteroscope and other elements. FIG. 2B shows that after simultaneously rapidly injecting the component A and the component B (blue) into renal calyces where the stone fragments are located through an operating passage of the flexible ureteroscope, 1 ml of normal saline is injected. FIG. 2C shows the gel wrapping the stone fragments after waiting for 3-5 seconds.

### Fourth Embodiment

This embodiment mainly studies the size range of the human stones that are able to be removed from the human body by the cooperation of the gel and the 12 Fr of UAS.

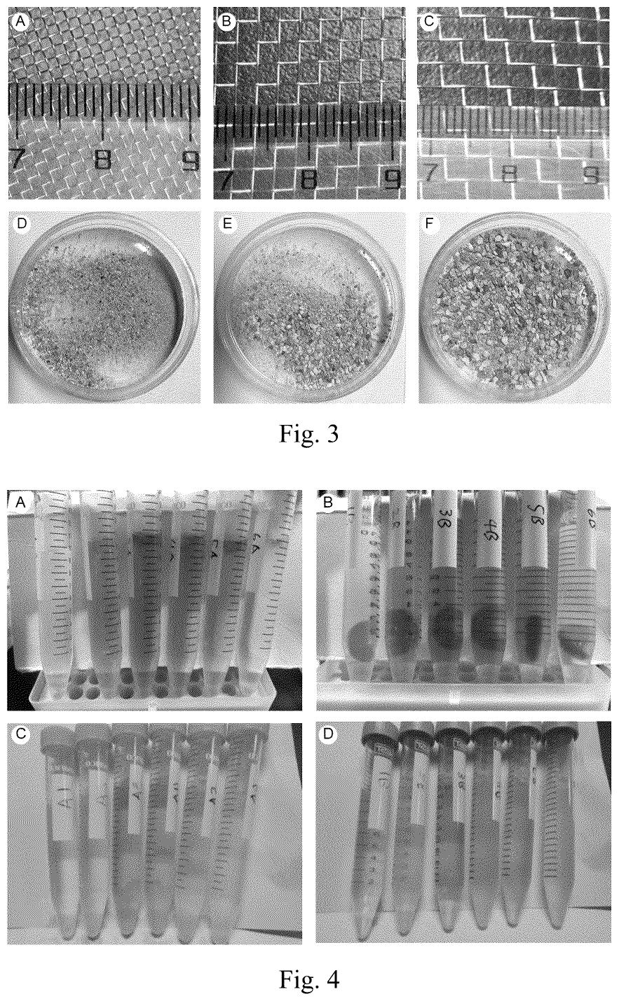

The experimental process comprises steps of:
(1) grinding and naturally-drying urinary stone specimens (mainly containing calcium oxalate monohydrate and calcium oxalate dihydrate) which are clinically collected, and screening out stones with a particle size less than or equal to 1 mm, 2 mm and 3 mm by three metallic mesh screenings with a mesh size of 1 mm, 2 mm and 3 mm, respectively, as shown in FIG. 3; and
(2) performing lithotomy by the gel which is formed by the dilute solutions of two components with a dilution multiple of 32 times according to the second embodiment of the present invention, wherein:
experimental results show that the stones with the particle size less than or equal to 1 mm and 2 mm, which are wrapped by the fibrin gel, are able to be removed from the human body through the 12 Fr of UAS by negative pressure suction or the stone extractor; and moreover, the stones with the particle size less than or equal to 3 mm, which are wrapped by the fibrin gel, are able to be removed from the human body by the stone extractor, but the blockage often occurs when these stones are removed from the human body by negative pressure suction (as shown in Table 1).

TABLE 1
Performing lithotomy with the fibrin
gel (through the 12 Fr of UAS)

| | | | |
|---|---|---|---|
| Particle size of stones | ≤1 mm | ≤2 mm | ≤3 mm |

Negative pressure suction

| | | | |
|---|---|---|---|
| The number of successful suction/The | 10/10 | 10/10 | 3/10 |
| total number of suction (%) | (100%) | (100%) | (30%) |
| The number of blockage/The | 0/10 | 0/10 | 7/10 |
| total number of suction (%) | (0) | (0) | (70%) |

Grabbing with the stone extractor

| | | | |
|---|---|---|---|
| The number of successful grabbing/The | 10/10 | 10/10 | 10/10 |
| total number of grabbing (%) | (100%) | (100%) | (100%) |
| The number of blockage/The | 0/10 | 0/10 | 1/10 |
| total number of grabbing (%) | (0) | (0) | (10%) |

### Fifth Embodiment

This embodiment mainly studies the characteristic that the fibrin gel formed by the two components is able to naturally dissolve in urine and normal saline. The experimental process comprises steps of
immersing the tough and high-density fibrin gel formed by the undiluted stock solutions of the components A and B (wherein the component B is added with methylene blue), and the fibrin gel formed by the dilute solutions of the components A and B with a dilution multiple of 32 times (wherein the component B is also added with methylene blue) into healthy human urine at 37° C. respectively according to the second embodiment of the present invention.

Experimental results are shown in FIG. 4. FIG. 4A shows 6 parts of the fibrin gels formed by the dilute solutions of the components A and B with a dilution multiple of 32 times. FIG. 4B shows 6 parts of the fibrin gels formed by the undiluted stock solutions of the components A and B. FIG. 4C shows that after putting the 6 parts of fibrin gels in FIG. 4A into 1 part of normal saline and 5 parts of healthy human urine from left to right respectively, and standing in a water bath at 37° C. for 24 hours, the 6 parts of fibrin gels in FIG. 4A are all dissolved. FIG. 4D shows that after putting the 6 parts of fibrin gels in FIG. 4B into 1 part of normal saline and 5 parts of healthy human urine from left to right respectively, and standing in the water bath at 37° C. for 24 hours, the 6 parts of fibrin gels in FIG. 4B are all dissolved. These results indicate that all of the fibrin gels are able to naturally dissolve in healthy human urine.

### Sixth Embodiment

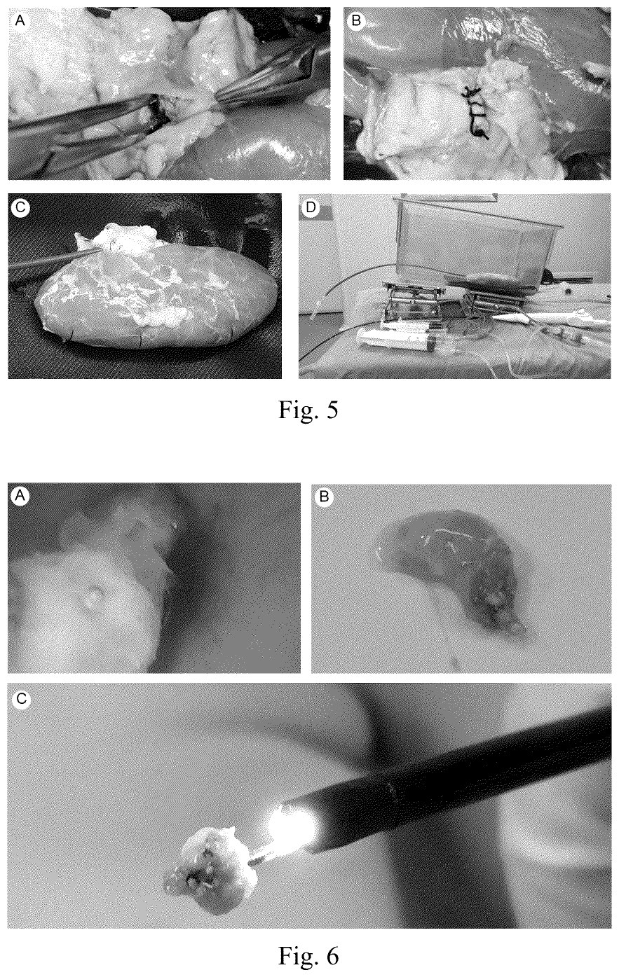

According to the sixth embodiment of the present invention, the fibrin gel is applied to an ex-vivo pig kidney for removing pre-implanted human stones and calculating the stone clearance rate. The experimental process comprises steps as follows.
(1) Prepare two different sizes (≤1 mm and ≤2 mm) of stone fragments, and construct a model of the ex-vivo pig kidney with the pre-implanted human stones, wherein:
main stone fragments produced in an actual lithotripsy usually contain stone fragments of a certain size range and dust, so the two different sizes of stone fragments are used for constructing the model of the ex-vivo pig kidney with the pre-implanted human stones;
constructing the model of the ex-vivo pig kidney with the pre-implanted human stones comprises:
preparing a fresh domestic pig kidney, making an incision with a length of 1 cm in a renal pelvis of the fresh domestic pig kidney, putting screened stone fragments and dust into the kidney via the incision (referring to FIG. 5A), closing the incision by continuous lock stitch suture with 2-0 silk yarns (referring to FIG. 5B), fixing the kidney and the renal pelvis on a silicon cushion with a thickness of 10 mm by interrupted suture through 2-0 silk yarns (referring to FIG. 5C), placing the kidney which is fixed on the silicon cushion into a plastic box with 37° C. normal saline, wherein the plastic box is supported by two lifting platforms with adjustable height, so as to ensure that the kidney, renal pelvis and ureter are in normal physiological and anatomical position; the UAS is sealed and fixed on a side of the plastic box (referring to FIG. 5D).
(2) Set the stone removal modes of the experimental group and the control group, wherein:
the stone removal mode of the experimental group is that the dilute solutions of the components A and B with a dilution multiple of 32 times according to the second embodiment of the present invention are used as raw materials, wherein the component B is added with methylene blue, the injection method shown in FIG. 2 according to the third embodiment of the present invention is adopted (the injection method comprises simultaneously injecting the components A and B of the fibrin gel through the operating passage of the flexible ureteroscope with three three-way stopcocks into the ex-vivo pig kidney, and adding a small amount of normal saline, and then removing the human stones by negative pressure suction and the stone extractor (referring to FIG. 6).

The stone removal mode of the control group is that the traditional negative pressure suction and stone extractor are used.

In order to avoid the influence of technical differences on the experimental results, the flexible ureteroscope is operated by a single surgeon (>1000 cases of RIRS experience) with rich experience in ureteroscope operation. The upper limit of the operation time of a single kidney operation is set to 30 minutes.

The amount of stones implanted per time is fixed in the control group and the experimental group. After the surgery, the collection system is opened, the residual stones are collected, and the stone clearance rate is indirectly calculated by a formula of:

$$Stone clearance rate = ( 1 - the mass of residual stones / the mass of implanted stones ) \times 100 \% .$$

Experimental results are shown in Table 2.
(1) For patients with ≤1 mm of stones, the operation time of the control group and the experimental group is 30:00 (minute, second), 30:00, 28:40 and 18:35, 13:20, 10:15, respectively. Accordingly, the stone clearance rate is 34.2%, 45.6%, 65.3% in the control group; 89.2%, 91.3%, 92.1% in the experimental group, respectively. Obviously, the stone clearance rate of the experimental group is significantly higher than that of the control group, p=0.0096.
(2) For patients with ≤2 mm of stones, the operation time of the control group and the experimental group is 30:00 (minute, second), 30:00, 30:00 and 13:42, 16:23, 8:28, respectively. Accordingly, the stone clearance rate is 15.2%, 29.6%, 16.3% in the control group; 92.9%, 85.4%, 95.0% in the experimental group, respectively. Obviously, the stone clearance rate of the experimental group is significantly higher than that of the control group, p=0.0002.

TABLE 2
Stone removal with gel in the ex-vivo pig kidney

| | Size of | | | Stone |
|---|---|---|---|---|
| Kidney | implanted | Whether the | Operation time | clearance |
| No. | stones (a) | gel is used | (minutes, seconds) | rate (b) |
| 1 | ≤1 mm | No | 30:00 | 34.2% |
| 2 | ≤1 mm | No | 30:00 | 45.6% |
| 3 | ≤1 mm | No | 28:40 | 65.3% |
| 4 | ≤1 mm | Yes | 18:35 | 89.2% |
| 5 | ≤1 mm | Yes | 13:20 | 91.3% |
| 6 | ≤1 mm | Yes | 10:15 | 92.1% |
| 7 | ≤2 mm | No | 30:00 | 15.2% |
| 8 | ≤2 mm | No | 30:00 | 29.6% |
| 9 | ≤2 mm | No | 30:00 | 16.3% |
| 10 | ≤2 mm | Yes | 13:42 | 92.9% |
| 11 | ≤2 mm | Yes | 16:23 | 85.4% |
| 12 | ≤2 mm | Yes | 8:28 | 95.0% |

Here, “a”: the total mass of air-dried stones implanted in a single kidney is about 100 mg.

“b”: the stone clearance rate is calculated by a formula of stone clearance rate=(1−the mass of residual stones/the mass of implanted stones)×100%.

### Seventh Embodiment

The gel used in this embodiment does not contain plasminogen provided by the second embodiment. The undiluted stock solutions of the component A which acts as the main body of the gel, and the component B which acts as the catalyst are as follows. In the component A, the total concentration of the fibrinogen and fibrin stabilizing factor is 3.8 mg/ml; in the component B, the concentration of the thrombin is 20 IU/ml, the concentration of Ca²⁺ is 5 mM.

The undiluted stock solutions of the components A and B are diluted by the dilution method provided by the step (2) of the second embodiment of the present invention.

The dilute solutions of the components A and B with a dilution multiple of 8 times, 16 times or 32 times according to this embodiment, are injected into the vial containing 10 ml of normal saline and a small amount of <2 mm of human stone fragments at the bottom of the vial by the third method of the third embodiment, respectively. In spite that according to the above third to sixth embodiments, the preferable dilution multiple is 32 times, this embodiment aims at observing whether the gel, formed by the dilute solutions of the components A and B with a lower dilution multiple (a higher concentration), block the operating passage of the flexible ureteroscope with a diameter in a range of 1-1.2 mm, or observing whether the blockage is conveniently and effectively dredged after blockage by existing materials (such as a guide wire with a diameter in a range of 0.8 to 1 mm, or a stone extractor with a diameter in a range of 0.8 to 1 mm), or by a simple method which comprises dredge the blockage by directly injecting normal saline.

Experimental results show that the dilute solutions of the components A and B with the dilution multiple of 8, 16 and 32 times are able to form the gel in the liquid environment of normal saline, and simultaneously, the operating passage of the flexible ureteroscope is not blocked. Even if no additional normal saline is added to flush the operating passage, it is convenient to use the wire or the stone extractor for dredging the operating passage. Moreover, the gel, formed by the dilute solutions of the components A and B with the dilution multiple of 8, 16 and 32 times in the liquid environment of normal saline, is able to effectively adhere to and wrap<2 mm of stone fragments; the gel-stone complex formed by adhering and wrapping stones with the gel has ideal plasticity and toughness, and is able to be removed from the human body through the UAS with the inner diameter in a range of 3-4 mm by the stone extractor.

### Eighth Embodiment

According to this embodiment of the present invention, the fibrin gel is applied to an ex-vivo pig kidney for removing pre-implanted human stones. In the state of continuous circulation of water, the formation state and characteristics of the gel may change. Therefore, this embodiment focuses on evaluating the characteristics of gel adhering to and wrapping stones under the condition of maintaining the 5, 10 or 20 ml/min of normal saline circulation flow (which is consistent with the perfusion flow used in actual surgery), the properties of the formed gel-stone complex and whether the gel-stone complex is removed from the human body through the UAS, wherein the dilute solutions of the components A and B have the dilution multiple of 8, 16 or 32 times.

The components A and B used in this embodiment are the same as those in the seventh embodiment.

The experimental method of this embodiment is basically the same as that of the seventh embodiment. The difference is that in this embodiment, in the vial containing 10 ml of normal saline and a small amount of <2 mm of human stone fragments at the bottom of the vial, the 5, 10 or 20 ml/min of normal saline circulation flow is maintained, that is, normal saline is continuously injected into the vial at a flow rate of 5, 10 or 20 ml/min by a peristaltic pump with constant flow perfusion function.

Experimental results show that at the circulation flow of 5, 10 or 20 ml/min, the gel formed by the dilute solutions of the components with the dilution multiple of 8 times is used to adhere to and wrap the stones for obtaining the gel-stone complex, the obtained gel-stone complex has the best characteristics that it is tough enough and not easy to be broken, and is able to be removed from the human body through the UAS by the stone extractor; the gel, formed by the dilute solutions of the two components with the dilution multiple of 8 times, is able to adhere to and wrap the stones for obtaining the gel-stone complex, but the obtained gel-stone complex is easy to be broken while being removed from the human body through the UAS by the stone extractor, which results in more times of grasping; the gel formed by the dilute solutions of the two components with the dilution multiple of 32 times is unable to sufficiently adhere to and wrap the stones.

### Ninth Embodiment

This embodiment is basically the same as the eighth embodiment. The difference is that the gel, which is formed by the dilute solutions of the two components with the dilution multiple of 8 and 10 times respectively, is used in this embodiment, wherein the dilution factor is for the undiluted stock solution in the seventh embodiment. Dilution of 10 times is more convenient for dispensing calculation in practical clinical applications.

The experimental method of this embodiment is the same as that of the eighth embodiment.

Experimental results show that at the circulation flow of 5, 10 or 20 ml/min, the gels formed by the dilute solutions of the two components with the dilution multiple of 8 and 10 times are used to adhere to and wrap the stones for obtaining the gel-stone complexes respectively, the obtained gel-stone complexes have the same characteristics that they are tough enough and not easy to be broken, and are able to be removed from the human body through the UAS by the stone extractor. Therefore, it is obvious that in practical clinical applications, dilution of 8 times, dilution of 10 times, or any value between the above two dilution times is able to be used.

### Tenth Embodiment

The two components A and B of the gel in this embodiment are basically the same as the two components A and B with the dilution multiple of 10 times in the ninth embodiment. The difference is that the component B is added with 0, 20, 60, 120 or 240 μg/ml of methylene blue in this embodiment, that is, the undiluted stock solutions of the component A which acts as the main body of the gel and the component B which acts as the catalyst are as follows.

In the component A, the total concentration of the fibrinogen and fibrin stabilizing factor is 3.8 mg/ml; in the component B, the concentration of the thrombin is 20 IU/ml, the concentration of Ca²⁺ is 5 mM. The dilute solution of the component B with the dilution multiple of 10 times is added with methylene blue, so that the final concentration of methylene blue in the component B is 0, 20, 60, 120 and 240 μg/ml respectively.

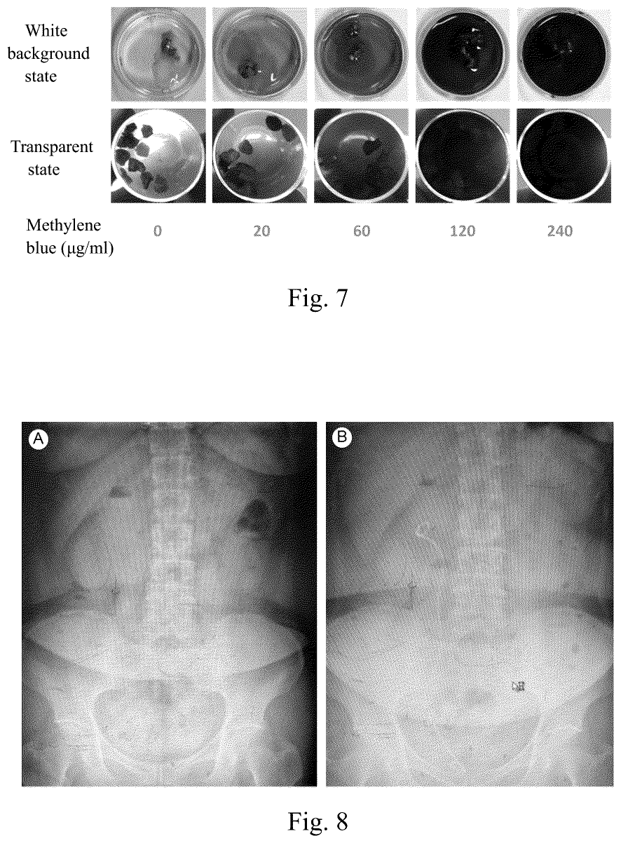

Experimental results show that the fibrin gel, prepared by the undiluted stock solutions of the components A and B, is milky; and the gel, prepared by the dilute solutions of the components A and B, is almost transparent and difficult to distinguish in normal saline. The dilute solution of the component B added with methylene blue effectively highlights the gel to blue in normal saline without affecting the operating field and the display of the wrapped stones (referring to FIG. 7).

### Eleventh Embodiment

According to the eleventh embodiment of the present invention, the porcine fibrin sealant produced by Bioseal Biotech, Guangzhou Province, China, which is the same as the gel provided by the present invention in composition principle and has been widely used in hemostasis of surgical wounds, is diluted by normal saline, and then is added with methylene blue injection which has the concentration of 10 mg/ml and is produced by Jumpcan, Jiangsu Province, China, and is finally used for URL in patients with renal and ureteral stones.

Patent information: due to right hydronephrosis, a 53-year-old female, hospitalized No. D01280643, is admitted to the Department of Urology, the First Affiliated Hospital of Nanchang University on Mar. 8, 2021; plain abdominal radiograph (DX202103081630) shows that right ureteral stone, about 0.8 cm×1.8 cm in size (referring to FIG. 8a); the patient undergoes the URL (right) on Mar. 9, 2021.

Surgical Material: porcine fibrin sealant produced by Bioseal Biotech, Guangzhou Province, China; methylene blue (111598, 10 mg/ml, Jumpcan, Jiangsu Province, China); guide wire (HWS-035150, COOK® MEDICAL LLC, Indiana, USA), 12/14 Fr of UAS (female: FUS-120035, COOK® MEDICAL LLC, Indiana, USA); flexible ureteroscope (USCOPE, PUSEN Medical, Guangdong Province, China); stone extractor (NTSE-022115-UDH, COOK® MEDICAL LLC, Indiana, USA); ureteral stent (Universa® Firm, UFI-626-R, COOK® MEDICAL LLC, Indiana, USA), three-way stopcock (BD Connecta, 394605, BD Medical, Helsingborg, Sweden), holmium laser fiber (SlimLine SIS 200 Laser Fiber, Lumenis), holmium laser host (versapulse powersuite 100W Holmium Laser System, Lumenis, Yokneam, Israel), high pressure water pump (medical pressurizer RXJ-I, Tonglu Rex Medical Instrument Co., Ltd., Hang Zhou, China).

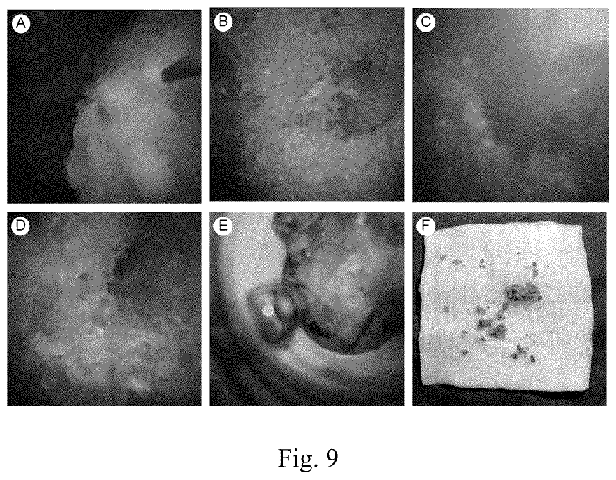

Surgical methods: FIG. 9A shows that the 12/14 Fr of UAS is indwelled by conventional method, intrarenal conditions are explored by the flexible ureteroscope, the stones are located and pulverized by pop-dusting with laser; FIG. 9B shows that the stones are pulverized into <2 mm of stone fragments; preparing a gel: add 45 ml of normal saline into 5 ml of the component A of the porcine fibrin adhesive, 45 ml of normal saline into 5 ml of the component B and 0.4 ml of methylene blue of the porcine fibrin adhesive; preparing a gel injection system: there entrances of three three-way stopcocks at a side thereof are connected with three syringes respectively, wherein there is normal saline in a top syringe, there is the component A in a middle syringe, and there is the blue component B in a bottom syringe; the common water entry passage is removed and instead of the gel injection system; the dilute solutions of the components A and B are simultaneously injected into the designated area (referring to FIG. 9C), and then 5 ml of normal saline is injected for flushing the operating passage within 5 seconds; and then, the gel injection system is removed and instead of the common water entry passage; the position of gel is observed (as shown in FIG. 9D), and the gel is removed by NCircle® or NGage® stone extractor, referring to FIG. 9E; the pelvis and each calyx are checked and all gels are removed from the human body.

Surgical effect: referring to FIG. 9f, a composition gel adhering to a large number of stone fragments is removed intraoperatively, and these stone fragments are unable not be removed intraoperatively without the use of the composition gel and had to be excreted by the patient after surgery. On the first day after surgery, CT examination of the middle and lower abdomen shows no significant residual stones.

It should be noted that the above embodiments are used only to illustrate the technical schemes of the present invention and not the limitation. In spite that the present invention is described in detail with reference to the foregoing embodiments, those skilled in the art should understand that the technical schemes recorded in the foregoing embodiments may be modified or some or all of the technical features thereof may be replaced equivalently. These modifications or replacements do not make the essence of the corresponding technical schemes out of the protective scope of the technical scheme of each embodiment of the present invention.

1: A composition gel for improving residual stone fragments after lithotripsy, the composition gel comprising component A and component B, wherein:
the component A contains fibrinogen and a fibrin stabilizing factor;
the component B contains thrombin;
at least one of the component A and the component B contains Ca²⁺.

2: The composition gel according to claim 1, wherein the at least one of the component A and the component B contains plasminogen; a concentration of the plasminogen is in a range of 1/256 to 1 of a concentration range of 0.1 mg/ml-1 mg/ml.

3-10. (canceled)

11: The composition gel according to claim 2, wherein the concentration of the plasminogen is in a range of 1/64 to 1 of the concentration range of 0.1 mg/ml-1 mg/ml.

12: The composition gel according to claim 2, wherein the concentration of the plasminogen is in a range of 1/45 to 1/20 or 1/32 to ⅛ of the concentration range of 0.1 mg/ml-1 mg/ml.

13: The composition gel according to claim 2, wherein the concentration of the plasminogen is in a range of 1/35 to 1/25 or 1/10 to ⅛ of the concentration range of 0.1 mg/ml-1 mg/ml.

14: The composition gel according to claim 2, wherein the concentration of the plasminogen is in a range of 1/32 or 1/10 of the concentration range of 0.1 mg/ml-1 mg/ml.

15: The composition gel according to claim 1, wherein the at least one of the component A and the component B contains a non-cyto toxic colouring agent.

16: The composition gel according to claim 2, wherein the at least one of the component A and the component B contains a non-cyto toxic colouring agent.

17: The composition gel according to claim 14, wherein the at least one of the component A and the component B contains a non-cyto toxic colouring agent.

18: The composition gel according to claim 15, wherein the colouring agent is methylene blue, chlorophyll or indocyanine green.

19: The composition gel according to claim 17, wherein the colouring agent is methylene blue, chlorophyll or indocyanine green.

20: The composition gel according to claim 18, wherein the colouring agent is the methylene blue with a concentration in a range of 20.0 μg/ml to 60.0 μg/ml.

21: The composition gel according to claim 19, wherein the colouring agent is the methylene blue with a concentration in a range of 20.0 μg/ml to 60.0 μg/ml.

22: The composition gel according to claim 17, wherein both the fibrinogen and the fibrin stabilizing factor are derived from human or animal blood or blood products.

23: The composition gel according to claim 15, wherein the component A further contains normal saline; the fibrinogen and the fibrin stabilizing factor are diluted in the normal saline.

24: The composition gel according to claim 17, wherein the component A further contains normal saline; the fibrinogen and the fibrin stabilizing factor are diluted in the normal saline.

25: The composition gel according to claim 24, wherein both the thrombin and the plasminogen are derived from human or animal blood or blood products.

26: The composition gel according to claim 24, wherein the component B further contains normal saline, wherein the thrombin is diluted in the normal saline.

27: The composition gel according to claim 25, wherein the component B further contains normal saline, wherein the thrombin is diluted in the normal saline.

28: The composition gel according to claim 1, wherein:
a total concentration of the fibrinogen and the fibrin stabilizing factor of the component A is in a range of 1/256 to 1 of a concentration range of 1.8 to 3.8 mg/ml;
a concentration of the thrombin of the component B is in a range of 1/256 to 1 of a concentration range of 15.0 to 21.7 IU/ml;
a ratio of an amount of the thrombin of the component B to a total amount of the fibrinogen and the fibrin stabilizing factor of the component A is in a range of (15.0-21.7) IU: (1.8-3.8) mg;
in the at least one of the component A and the component B, a concentration of Ca²⁺ is in a range of 1/256 to 1 of a concentration of 5 mM.
